(19) United States
(12) Patent Application Publication
(10) Pub. No.: US 2015/0250879 A1
(43) Pub. Date: Sep. 10, 2015

(54) TEST FOR HEMOLYTIC POTENTIAL OF PHARMACEUTICAL PRODUCTS AND FORMULATIONS FOR RISK MINIMIZATION

(71) Applicant: OCTAPHARMA AG, Lachen (CH)

(72) Inventors: Andrea Heger, Vienna (AT); Tor-Einar Svae, Vienna (AT); Juergen Roemisch, Vienna (AT); Alfred Zoechling, Vienna (AT)

(73) Assignee: OCTAPHARMA AG, Lachen (CH)

(21) Appl. No.: 14/431,498

(22) PCT Filed: Sep. 26, 2013
(86) PCT No.: PCT/EP2013/070111

§ 371 Date: Mar. 26, 2015

(30) Foreign Application Priority Data
Sep. 27, 2012 (EP) 12186382.3
Apr. 10, 2013 (EP) 13163162.4

(51) Int. Cl. A61K 47/18; C07K 16/18; G01N 33/49

(52) U.S. Cl. CPC A61K 47/183; G01N 33/49; C07K 16/18

(57) ABSTRACT

A non-hemolytic acidic composition comprising an immunoglobulin selected from immunoglobulin A, immunoglobulin G or immunoglobulin M at a pH value in the range of 4.65 to 5.7 comprising one or more excipients selected from amino acids, sugars and sugar alcohols in a concentration range of 100-350 mmol/l, in particular 200-350 mmol/l, characterized in that it displays an optical density (OD) in the range of 0.14 to −0.1 when the OD is determined in a spectrophotometer at 414 nm at a film thickness of 0.825 mm; and its use in an assay for determination of the hemolytic potential of intravenously applied pharmaceuticals and compositions and preparations displaying a reduced potential of hemolysis when being applied intravenously.

This invention claims priority of the European patent applications EP 12 186 382, dated 27 Sep. 2012 and EP 13 163 162, dated 10 Apr. 2013.

This invention provides an in-vitro test to assess the hemolytic potential of intravenously applied pharmaceutical products as well as formulations for minimizing the risk of hemolysis after application of said products.

With the introduction of pharmaceuticals, in particular immunoglobulins, formulated in a clearly acidic pH range being applied the numbers of some adverse reactions increased. Administration of immunoglobulin concentrates, e.g. in particular immunoglobulin G for intravenous application—but not limited to—, may lead to mild-to-moderate adverse events, including low-grade fever, headache, malaise, nausea, myalgia, and urticaria. Less common but serious and potentially fatal adverse events include thrombotic complications, acute renal failure, anaphylaxis, hemolysis, and aseptic meningitis [Daniel G W, Menis M, Sridhar G, Scott D, Wallace A E, Ovanesov M V, Golding B, Anderson S E, Epstein Y, Martin D, Ball R, Izurietta H S. Immune globulins and thrombotic adverse events as recorded in a large administrative database in 2008 through 2010. Transfusion. 2012]. Examples of literature discussing hemolytic anemia after intravenous immunoglobulin treatment are Zohra Daw et al. [Hemolytic transfusion reactions after administration of intravenous immune (gamma) globulin: a case series analysis. Transfusion, 2008, Vol.48, 1598-1601], Joseph Kahwaji et al.[Acute Hemolysis After High-Dose Intravenous Immunoglobulin Therapy in Highly HLA Sensitized Patients. Clin J Am Soc Nephrol, 2009, 1-5] and Roberta Berard et al. [Hemolytic anemia following intravenous immunoglobulin therapy in patients treated for Kawasaki disease: a report of 4 cases. Pediatric Rheumatology Online Journal, 2012, 10:10]. In recent years, increased numbers of hemolysis were reported in patients after administration of IVIG's with acidic formulation. It is known from U.S. 2005/154165 A1 that (meth)acrylic copolymers induce hemolysis of more than 60% at pH 5.5 and less than 5% at pH 7.4 determined by a cytotoxicity test with human red blood cells which is based on cell count.

### SUMMARY OF THE INVENTION

One object of the present invention relates to acidic compositions of intravenously applied pharmaceuticals capable of eliminating or at least minimizing the risk of triggering hemolysis. It was now surprisingly found that some compounds already used as stabilizers, such as sugars, sugar alcohols or amino acids, can also be used for reduction of the hemolytic potential of such preparations.

Said hemolytic potential can be assessed by another object of the present invention relating to an in-vitro hemolysis assay of based on a photometric determination of red blood cell (RBC) lysis at 414 nm. While prior art assays use erythrocytes sensitized with antibodies and covered with complement component, e.g. WO 2004/007553 A1, it was surprisingly found that it is sufficient for the assay of the present invention to utilize RBC's which are neither sensitized with antibodies nor covered with complement component.

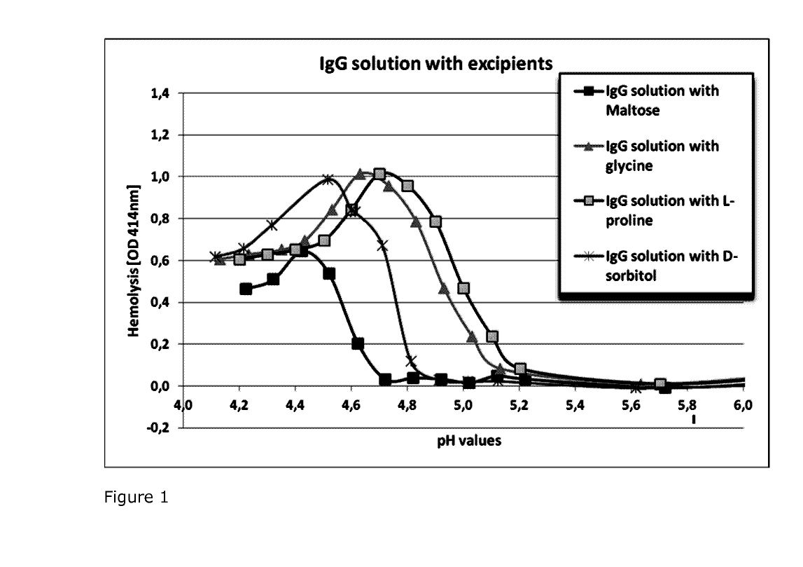

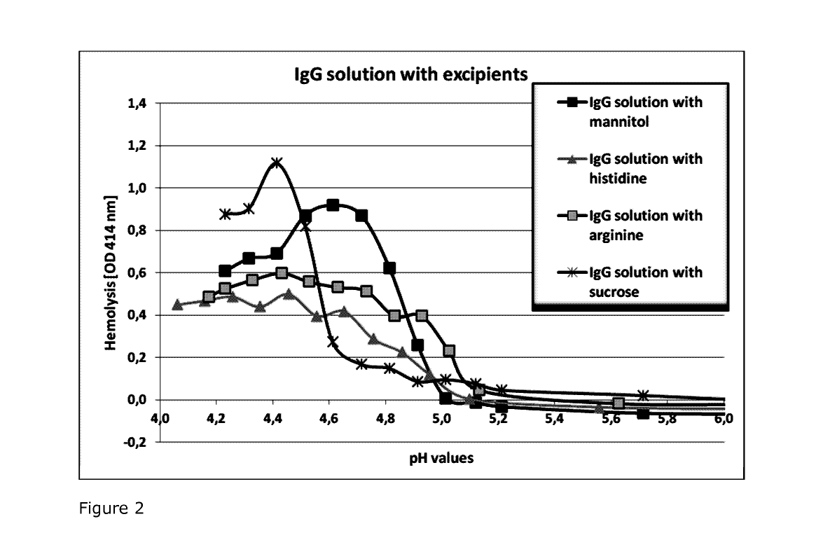

FIGS. 1 and 2 display the influence of immunoglobulins with various excipients on hemolysis in dependence of pH-value.

FIG. 1 displays graphically the data provided in table 2.

FIG. 2 displays graphically the data provided in table 3.

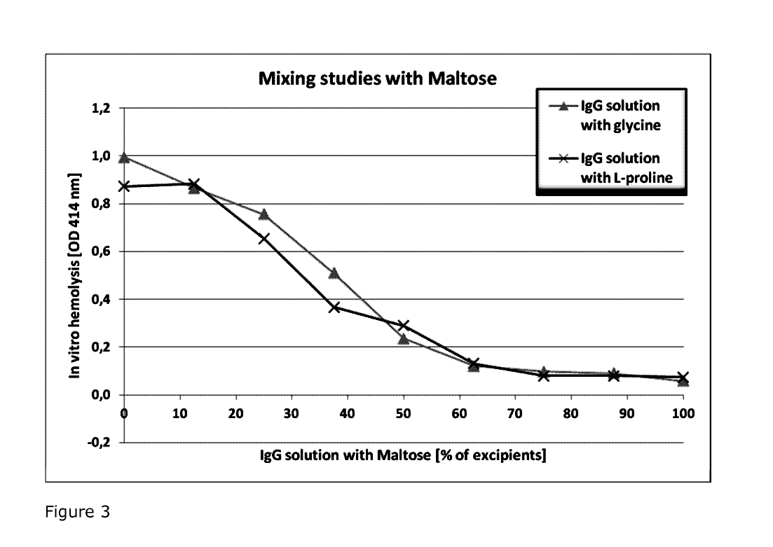

FIG. 3 displays the influence of immunoglobulins with a mixture of excipients on hemolysis in dependence of pH-value and displays the result of a mixed composition wherein the content of glycine respectively L-proline was progressively replaced by maltose while other parameters like pH were kept constant.

According to the invention there is provided a non-hemolytic acidic composition comprising an immunoglobulin selected from immunoglobulin A, immunoglobulin G or immunoglobulin M at a pH value in the range of 4.65 to 5.7 comprising one or more excipients selected from amino acids, sugars and sugar alcohols in a concentration range of 100-350 mmol/l, in particular 200-350 mmol/l, characterized in that it displays an optical density (OD) in the range of 0.14 to -0.1 when the OD is determined in a spectrophotometer at 414 nm at a film thickness of 0.825 mm.

In an embodiment of the present invention the excipients are selected from glycine, L-proline, arginine, histidine, D-sorbitol, mannitol, maltose and sucrose.

In another embodiment the non-hemolytic acidic composition of the invention may have an osmolality in the range of 200-400 mOsmol/kg, in particular 230-350 mOsmol/kg.

Subject matter of the present invention is also a pharmaceutical composition comprising the non-hemolytic acidic composition of the invention and a pharmaceutically acceptable carrier.

In an embodiment of the pharmaceutical composition of the invention the immunolgobulin is immunoglobulin G.

In another embodiment of the pharmaceutical composition of the invention the immunoglobulin G is formulated for subcutaneously, intravenously or intramuscularly administration.

A further subject matter of the present invention is a pharmaceutical composition of the invention for the treatment of immune deficiencies such as X-linked agammaglobulinemia, hypogammaglobulinemia, acquired compromised immunity conditions (secondary immune deficiencies) featuring low antibody levels, autoimmune diseases, e.g. immune thrombocytopenia ITP, and inflammatory diseases, e.g. Kawasaki disease, chronic inflammatory demyelinating polyneuropathy (CIDP) and neurological diseases, e.g. multifocal motor neuropathy, myasthenia gravis and multiple sclerosis.

Still another subject matter of the present invention is the use of a nonhemolytic acidic composition of the invention in an assay for determination of the hemolytic potential of intravenously applied pharmaceuticals comprising the steps of:
a) providing a test sample containing a pharmaceutical at a concentration of 5-23% w/v solubilized in water for injection (WFI);
b) mixing of equal volumes of the test sample and a red blood cell dispersion with the provision that the red blood cells are not sensitized;
c) incubation of the mixture for 8-24 hours, in particular 12-18 hours, at 20-25° C. and centrifugation at 20.000×g for 5 minutes;
d) measurement of the OD of 100 μl of the supernatant obtained in step c) at a film thickness of 0.825 mm, wherein the non-hemolytic acidic composition comprises an immunoglobulin selected from immunoglobulin A, immunoglobulin G or immunoglobulin M at a pH value in the range of 4.65 to 5.7 comprising one or more excipients selected from amino acids, sugars and sugar alcohols in a concentration range of 100-350 mmol/l, in particular 200-350 mmol/l, wherein the non-hemolytic acidic composition displays an optical density (OD) in the range of 0.14 to -0.1 when the OD is determined in an spectrophotometer at 414 nm.

### DETAILED DESCRIPTION OF THE INVENTION

The assay used for the risk assessment comprises mixing of immunoglobulin samples, optionally containing an excipient to prevent hemolysis, with a RBC dispersion and incubation over night, in particular for 8-24 hours, in particular 1218 hours, at room temperature 20-25° C. The incubated samples were centrifuged the following day for 5 minutes at room temperature and 20,000×g. Subsequently, 100 μl of the supernatant was placed in flat bottomed 96 well plates (film thickness 0.825 mm) and the absorbance was measured at 414 nm (which is the extension maximum of exoplasmatic hemoglobin) using a spectrophotometer. The extent of hemolysis correlates with the absorbance peak at 414 nm. Thermally inactivated IgG was used as negative control sample with the optical density (OD) of the control sample being <0.1.

The RBC dispersions used were either prepared by washing RBC concentrates three times using 0.9% w/v NaCl, in-between centrifugation at 3000×g and disposal of each supernatant, or by separation of RBCs from fresh whole blood by centrifugation for 10 minutes at +4° C. and 3000×g and use of the same washing procedure as for RBC concentrates. Thus washed RBCs were brought to 0.5% (v/v) in 0.9% w/v NaCl and used for the assay.

An exemplary determination of hemolysis potential of an immunoglobulin was achieved by mixing 200 μl of a 10% w/v IgG (immunoglobulin diluted in water for injection (WFI)) test sample with 200 μl of RBC dispersion and incubation over night at room temperature. The incubated samples were centrifuged the following day for 5 minutes at room temperature and 20,000×g. Subsequently, 100 μl of the supernatant was placed in 96 well plates and the absorbance was measured at 414 nm.

Various tests of this kind were performed in the pH range of 4.0 to 6.0 with different excipients for prevention of hemolysis. It was apparent, that hemolysis occurred predominantly in the pH range of 4.0 to 5.7. Determination of pH values was performed with solutions containing 1 mg immunoglobulin in 1 ml physiological (0.9%) NaCl-solution, i.e. 0.1% w/v immunoglobulin.

IgG test samples were prepared from Immunoglobulin G (IgG) solutions of known initial concentration (w/v), the immunoglobulin being dissolved in WFI, by adjusting the IgG concentration to 10% w/v by addition of relevant amounts of WFI. The determination of the hemolytic potential of IgG solutions of a known lower concentration than 10% should be performed at adequate film thickness in order to compensate concentration differences.

A variation of RBCs regarding blood type revealed no significant difference between the results. It was thus decided to proceed with RBCs derived from donors of blood groups “A, Rh+”, “A, Rh−”, “AB, Rh+”, “O, Rh+” and “O, Rh−” for further examinations. A mean value was calculated for the OD and displayed in the figures for the sake of clarity.

A second aspect of this application is the provision of non-hemolytic acidic compositions and non-hemolytic acidic pharmaceutical preparations which are capable of suppression of hemolysis caused by pharmaceuticals -in order to increase patient safety. Said pharmaceuticals may in principal be intended for subcutaneous (s.c.), intravenous (i.v.) or intramuscular (i.m.) application of which those applied intravenously are of particular interest and of even more particular interest are intravenously applied immunoglobulins.

The assay described above was used for controlling the efficiency of various excipients in said task. The only variation performed was provision of the initial IgG solution described above which additionally contained at least one of the excipients selected from amino acids, sugars and sugar alcohols, in particular glycine, L-proline, arginine, histidine, D-sorbitol, mannitol, maltose and sucrose in a concentration range of 100-350 mmol/l, in particular in the range of 200-350 mmol/l. For example, an initial IgG solution of 20% containing 200 mmol/l of glycine was diluted 1:2 for provision of the 10% IgG test sample. Consequently, this test sample contained 100 mmol/l of glycine. As the initial concentration of excipients is relevant for the purpose of this application, all given concentrations refer to this initial concentration as it would be present in a pharmaceutical composition.

### EXAMPLE 1

The OD of a 10% IgG solution containing glycine (225 mmol/l) with an osmolality of 240 mOsmol/kg was determined with the above described assay to be 0.76 at a pH of 4.8.

### EXAMPLE 2

The OD of a 10% IgG solution containing glycine (250 mmol/l) with an osmolality of 290 mOsmol/kg was determined with the above described assay to be 0.74 at a pH of 4.8.

### EXAMPLE 3

The OD of a 10% IgG solution containing glycine (225 mmol/l) with an osmolality of 240 mOsmol/kg was determined with the above described assay to be 0.091 at a pH of 5.1.

Multiple experiments were performed with excipients of various initial concentrations in the range of 100-350 mmol/l. The excipients were tested within the range of initial concentration as indicated in table 1. The range of most positive excipient effect was found to be 200-350 mmol/l while osmolality was in the range of 200-400 mOsmol/kg, in particular 230-350 mOsmol/kg. The influence of osmolality on OD was negligible when the same excipient was used in samples with a pH of 4.7 to 6.2 and as long as osmolality was kept in the range of 230-350 mOsmol/kg. In contrast, significant differences in OD were occasionally observed between samples when osmolality was outside the range of 200-400 mOsmol/kg although the other parameters, i.e. immumoglobulin concentration, excipient concentration and pH were identical. This observation depended additionally on the pH value of the sample. For example, it was observed that two identical samples except for osmolality, one having 310 mOsmol/kg and the other 500 mOsmol/kg, displayed almost no OD difference at a pH value of 5.2, but they displayed an OD difference of about 0.5 at a pH of 4.9. As a consequence, data disclosed in tables 2 and 3 were obtained from samples having an osmolality within the range of 230-350 mosmol/kg.

TABLE 1

| Excipient | Range [mmol/l] |
|---|---|
| Glycine | 160-260 |
| L-Proline | 210-290 |
| Arginine | 250 |
| Histidine | 250 |
| Succrose | 150-290 |
| Maltose | 200-350 |
| Mannitol | 275 |
| D-sorbitol | 275 |

Compositions of OD 0.35 to −0.1 displayed strongly reduced or no hemolysis, in particular compositions with an OD of 0.14 to −0.1 were deemed exceptionally save as the negative control samples displayed OD's less than 0.10.

Pharmaceutical preparations of interest are preparations containing immunoglobulins selected from Immunoglobulin A, Immunoglobulin G or Immunoglobulin M, in particular at concentrations of 5-23% w/v.

Pharmaceutical products according to the present invention may also comprise other excipients like detergents, e.g. Polysorbate, for other reasons.

Pharmaceutical products, in particular immunoglobulin concentrates for intravenous, subcutaneous or intramuscular application, formulated according to the present invention are exceptionally useful in the treatment of Immune deficiencies such as X-linked agammaglobulinemia, hypogammaglobulinemia (primary immune deficiencies), acquired compromised immunity conditions (secondary immune deficiencies) featuring low antibody levels, autoimmune diseases, e.g.

Immune thrombocytopenia ITP, inflammatory diseases, e.g. Kawasaki disease, chronic inflammatory demyelinating polyneuropathy (CIDP) and neurological diseases, eg. multifocal motor neuropathy, myasthenia gravis and multiple sclerosis as adverse reactions are minimized.

TABLE 2

| [OD 414 nm] | | [OD 414 nm] | | [OD 414 nm] | | [OD 414 nm] | |
|---|---|---|---|---|---|---|---|
| Glycine | | Maltose | | D-Sorbitol | | L-Proline | |
| (225 mM) | | (290 mM) | | (275 mM) | | (250 mM) | |
| pH test | | pH test | | pH test | | pH test | |
| sample | mean | sample | mean | sample | mean | sample | mean |
| 4.1 | 0.606 | 4.2 | 0.464 | 4.1 | 0.618 | 4.2 | 0.606 |
| 4.2 | 0.625 | 4.3 | 0.509 | 4.2 | 0.658 | 4.3 | 0.629 |
| 4.3 | 0.684 | 4.4 | 0.645 | 4.3 | 0.767 | 4.4 | 0.650 |
| 4.4 | 0.737 | 4.5 | 0.537 | 4.5 | 0.987 | 4.5 | 0.694 |
| 4.5 | 0.905 | 4.6 | 0.204 | 4.6 | 0.841 | 4.6 | 0.844 |
| 4.6 | 0.911 | 4.7 | 0.032 | 4.6 | 0.831 | 4.7 | 1.014 |
| 4.7 | 0.931 | 4.8 | 0.039 | 4.7 | 0.672 | 4.8 | 0.957 |
| 4.8 | 0.760 | 4.9 | 0.033 | 4.8 | 0.119 | 4.9 | 0.784 |
| 4.9 | 0.370 | 5.0 | 0.016 | 4.9 | 0.030 | 5.0 | 0.467 |
| 5.0 | 0.146 | 5.1 | 0.049 | 5.0 | 0.021 | 5.1 | 0.235 |
| 5.1 | 0.091 | 5.2 | 0.034 | 5.1 | 0.024 | 5.2 | 0.083 |
| 5.6 | 0.007 | 5.7 | −0.007 | 5.6 | −0.008 | 5.7 | 0.010 |
| 6.1 | 0.026 | 6.2 | 0.024 | 6.1 | 0.008 | 6.2 | 0.047 |
| OD neg. | <0.1 | OD neg. | <0.1 | OD neg. | <0.1 | OD neg. | <0.1 |
| control | | control | | control | | control | |
| (thermally | | (thermally | | (thermally | | (thermally | |
| inactivated | | inactivated | | inactivated | | inactivated | |
| IgG) | | IgG) | | IgG) | | IgG) | |

Table 2 displays mean OD values of 10% IgG solutions composed with various excipients at various pH-values.

TABLE 3

| [OD 414 nm] | | [OD 414 nm] | | [OD 414 nm] | | [OD 414 nm] | |
|---|---|---|---|---|---|---|---|
| Mannitol | | Histidine | | Arginine | | Sucrose | |
| (275 mM) | | (250 mM) | | (250 mM) | | (290 mM) | |
| pH test | | pH test | | pH test | | pH test | |
| sample | (mean | sample | (mean) | sample | (mean) | sample | (mean) |
| 4.2 | 0.608 | 4.1 | 0.449 | 4.2 | 0.488 | 4.2 | 0.877 |
| 4.3 | 0.667 | 4.2 | 0.467 | 4.3 | 0.525 | 4.3 | 0.904 |
| 4.4 | 0.693 | 4.3 | 0.486 | 4.4 | 0.566 | 4.4 | 1.117 |
| 4.5 | 0.871 | 4.4 | 0.439 | 4.5 | 0.598 | 4.5 | 0.819 |
| 4.6 | 0.918 | 4.5 | 0.499 | 4.6 | 0.557 | 4.6 | 0.274 |
| 4.7 | 0.870 | 4.6 | 0.395 | 4.7 | 0.532 | 4.7 | 0.168 |
| 4.8 | 0.623 | 4.7 | 0.417 | 4.8 | 0.514 | 4.8 | 0.149 |
| 4.9 | 0.258 | 4.8 | 0.288 | 4.9 | 0.396 | 4.9 | 0.087 |
| 5.0 | 0.009 | 4.9 | 0.224 | 5.0 | 0.398 | 5.0 | 0.096 |
| 5.1 | −0.013 | 5.0 | 0.120 | 5.1 | 0.233 | 5.1 | 0.077 |
| 5.2 | −0.032 | 5.1 | 0.005 | 5.2 | 0.047 | 5.2 | 0.046 |
| 5.7 | −0.063 | 5.6 | −0.034 | 5.7 | −0.017 | 5.7 | 0.021 |
| 6.2 | −0.066 | 6.1 | −0.041 | 6.2 | −0.020 | 6.2 | −0.009 |
| OD neg. | <0.1 | OD neg. | <0.1 | OD neg. | <0.1 | OD neg. | <0.1 |
| control | | control | | control | | control | |
| (thermally | | (thermally | | (thermally | | (thermally | |
| inactivated | | inactivated | | inactivated | | inactivated | |
| IgG) | | IgG) | | IgG) | | IgG) | |

Table 3 displays OD values of 10% IgG solution composed with various excipients at various pH-values.

1. A non-hemolytic acidic composition comprising an immunoglobulin selected from immunoglobulin A, immunoglobulin G or immunoglobulin M at a pH value in the range of 4.65 to 5.7 comprising one or more excipients selected from amino acids, sugars and sugar alcohols in a concentration range of 100-350 mmol/l, characterized in that it displays an optical density (OD) in the range of 0.14 to −0.1 when the OD is determined in a spectrophotometer at 414 nm at a film thickness of 0.825 mm.

2. The non-hemolytic acidic composition of claim 1, wherein the excipients are selected from glycine, L-proline, arginine, histidine, D-sorbitol, mannitol, maltose and sucrose.

3. The non-hemolytic acidic composition of claim 1 having an osmolality in the range of 200-400 mOsmol/kg.

4. A pharmaceutical composition comprising the non-hemolytic acidic composition of claim 1 and a pharmaceutically acceptable carrier.

5. The pharmaceutical composition of claim 4, wherein the immunolgobulin is immunoglobulin G.

6. The pharmaceutical composition of claim 4, wherein the immunoglobulin G is formulated for subcutaneously, intravenously or intramuscularly administration.

7. The pharmaceutical composition of claim 4 for the treatment of immune deficiencies autoimmune diseases, inflammatory diseases, and/or neurological diseases.

8. An assay method for determining the hemolytic potential of intravenously applied pharmaceuticals comprising the steps of
a) providing a test sample containing a pharmaceutical at a concentration of 5-23% w/v solubilized in water for injection (WFI);
b) mixing equal volumes of the test sample and a red blood cell dispersion with the provision that the red blood cells are not sensitized;
c) incubating the mixture for 8-24 hours, at 20-25° C. and submitting it to centrifugation at 20.000×g for 5 minutes; and
d) measuring the OD of 100 μl of the supernatant obtained in step c) at a film thickness of 0.825 mm.

9. The composition of claim 1, wherein the amino acids, sugars and/or sugar alcohols are in a concentration range of 200-350 mmol/1.

10. The composition of claim 3, wherein the osmolality is in the range of 230-350 mOsmol/l.

11. The pharmaceutical composition of claim 7, wherein
the immune deficiency is X-linked agammaglobulinemia, hypogammaglobulinemia, or an acquired compromised immunity condition (secondary immune deficiency) featuring low antibody levels, and/or
the autoimmune disease is immune thrombocytopenia ITP, and/or
the inflammatory disease is Kawasaki disease or chronic inflammatory demyelinating polyneuropathy (CIDP), and/or
the neurological disease is multifocal motor meuropathy, myasthenia gravis or multiple sclerosis.

12. The assay method of claim 8, wherein in step c) the mixture is incubated for 12-18 hours.

13. The assay method of claim 8, wherein the pharmaceutical is a non-hemolytic acidic composition which comprises an immunoglobulin selected from immunoglobulin A, immunoglobulin G or immunoglobulin M at a pH value in the range of 4.65 to 5.7 and one or more excipients selected from amino acids, sugars and sugar alcohols in a concentration range of 100-350 mmol/l, wherein the non-hemolytic acidic composition displays an optical density (OD) in the range of 0.14 to −0.1 when the OD is determined in an spectrophotometer at 414 nm.

14. The assay method of claim 13, wherein the amino acids, sugars and/or sugar alcohols are in a concentration range of 200-350 mmol/l.
